(19) United States
(12) Patent Application Publication
(10) Pub. No.: US 2005/0181029 A1
(43) Pub. Date: Aug. 18, 2005

(54) Distributed drug dispensing matrix as a transdermal patch

(76) Inventors: Samir Mitragotri, Goleta, CA (US); Pankaj Karande, Goleta, CA (US)

Correspondence Address: FULBRIGHT & JAWORSKI L.L.P.;Robert Berliner, Twenty-Ninth Floor, 865 South Figueroa, Los Angeles, CA, 90017-2571, US

(21) Appl. No.: 10/782,155

(22) Filed: Feb. 18, 2004

(51) Int. Cl. A61K 9/70

(52) U.S. Cl. 424/448

(57) ABSTRACT

A transdermal drug delivery patch with an array of spatially organized reservoirs. The invention provides greater penetration of drug into the skin compared to current transdermal drug delivery systems, and facilitates the delivery of drugs that are presently refractory to transdermal delivery due to high molecular weight.

### BACKGROUND OF THE INVENTION

1. Field of the Invention

This invention relates generally to a transdermal drug delivery device.

2. Background Information

Drug delivery is a key factor that determines the commercial and therapeutic success of many drugs. It forms the driving force behind the development of many new drug delivery devices and formulations. Conventional drug delivery systems including oral administrations and injections find low patient compliance in addition to problems such as low availability of drugs in the targeted system due to fast bio-degradation.

Transdermal drug delivery is the administration of therapeutic agents across intact skin for systemic effects. It offers several main advantages over oral delivery including bypassing the hepatic first-pass, and maintaining the plasma drug level at a plateau over a long period of time. Transdermal applications, relative to other routes, are noninvasive; requiring the simple adhesion of a “patch” like device that holds the drug. Also since the skin offers a large (1-2 m²) and very accessible surface for drug delivery, one can have wide flexibility in choosing the location for application of this “patch”. In addition, transdermal drug delivery provides the potential for patient activated and patient modulated delivery, a feature rare in other drug delivery systems (1).

Despite these advantages transdermal drug delivery is restricted to only a handful of drugs (Scopolamine, Nitroglycerine, Clonidine, Estradiol, Fentanyl, Nicotine, Testosterone) due to the low permeability of the skin (2). The skin acts as a barrier between the organism and it's surroundings limiting molecular transport both from and into the body. Barrier properties of skin originate from its lipid bilayers that are located in the stratum corneum, the upper 10-15 microns of skin. The drug has to diffuse through 300 lipid bilayers in order to cross the stratum corneum (3). This limits the use of transdermal drug delivery route for several drugs that have high molecular weight. Several methods have been employed for modifying the skin properties that would enhance the drug penetration into the skin in sufficient quantities to achieve desired systemic effects (4).

Several patches are commercially available to deliver drugs mentioned above. These transdermal drug delivery systems fall into the following broad categories: 1. membrane permeation-controlled transdermal therapeutic system; 2. adhesive dispersion-type transdermal therapeutic system; and 3. matrix diffusion-controlled transdermal therapeutic system (2).

All of these drug delivery systems have a similar mode of dispensing the drug and differ only in the mode of controlling the rate of dispensing the drug or the way the drug is packed in the system. In all of these systems, the drug reservoir is in continuous contact with the entire skin area. Transport across the skin occurs heterogeneously. The molecular flux (number of molecules per unit area) varies substantially from one point to another over the entire area that is being used to deliver the drug.

Currently there is a need for a transdermal drug delivery system that would deliver a greater amount of drug than provided by presently employed transdermal drug delivery devices. In addition, transdermal delivery of drugs with high molecular weights is desirable.

### SUMMARY OF THE INVENTION

The present invention is a novel skin patch device for enhanced transdermal drug delivery. The invention provides a patch with several drug reservoirs arranged in a matrix. Dividing the area of contact with the skin into several smaller areas results in an increase in the amount of drug delivered. The invention can boost the delivery of drugs over that of current transdermal drug delivery systems, and facilitate the delivery of drugs that are presently refractory to transdermal delivery due to high molecular weight.

The novel features which are believed to be characteristic of the invention, both as to its organization and method of operation, together with further objects and advantages will be better understood from the following description about it's theory and working when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a schematic representation of skin held in diffusion cell apparatus. The skin swells due to hydration from an original thickness of h to h+Δh. The base radius stays constant at r. The swollen part of the skin can be treated as an oblate spheroid of major axis r and minor axis Δh.

FIG. 2 represents the dependence of theoretically calculated area strain, ε, on reservoir size r. Strain is seen to be non consequential to the transdermal delivery at large reservoir sizes (r>10 mm). The strain can be as high as 80% at a reservoir radius of 0.5 mm.

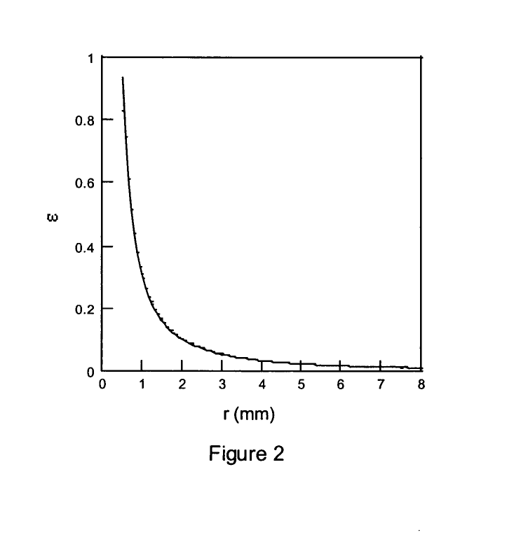

FIG. 3 is a schematic representation of a polycarbonate/teflon screening array to test the efficacy of a simulated distributed drug dispensing matrix patch. The figure shows the top view and the cross sectional view of the template with the model drug formulation and skin.

FIG. 4 is a graphic representation of the amount of mannitol delivered in to the skin as a function of inverse reservoir size in the assembly described in FIG. 3. This amount increases as the reservoir size decreases. This increase is sharp at reservoir size <10 mm but negligible at larger reservoir sizes. All enhancement factors have been normalized to the amount delivered at the largest reservoir size in the setup (16 mm). Error bars correspond to one standard deviation (n=4 to 9).

FIG. 5 is a schematic representation of a simulated distributed drug dispensing matrix patch made of a polymer (polyurethane) with varying reservoir sizes. The figure shows the top view and the cross sectional view of the template with the model drug formulation and skin in the receiver assembly.

FIG. 6 is a graphic representation of the permeability of skin to the formulation as a function of inverse reservoir size in the assembly described in FIG. 5. The permeability of skin increases as the reservoir size decreases. The dotted line represents the theoretically calculated area strain, ε at the reservoir sizes used in the experiments. The solid lines ε(γ+δγ) and ε(γ−δγ) represent the area strain calculated based on the uncertainty in the experimental determination of γ. All enhancement factors have been normalized to the enhancement obtained at the largest reservoir size in the setup (6 mm). The uncertainty in evaluating ε(γ) scales with the error bars on experimentally calculated enhancement factors represented by closed circles (n=3).

FIG. 7 is a schematic representation of a simulated distributed drug dispensing matrix patch made of agar gel drug discs with varying reservoir sizes. The figure shows the top view and the cross sectional view of the template with the model drug formulation on skin in a Franz diffusion cell.

FIG. 8 is a graphic representation of the permeability of the skin to the formulation at varying reservoir sizes in the assembly described in FIG. 7. Permeability increases with decreasing reservoir size. Error bars correspond to one standard deviation (n=5).

FIG. 9 shows the amount of mannitol delivered into the skin using a drug array constructed in accordance with this invention. Drug delivery enhancements obtained by using an array of agar gel disc reservoirs of varying reservoir sizes are shown as against a single large reservoir at a constant area fraction of 20%. The amount of mannitol delivered into the skin increases with a decrease in the reservoir size. All enhancements are obtained by normalizing the amount delivered into the skin at any particular reservoir size to that delivered at 16 mm. Error bars correspond to one standard deviation (n=5).

FIG. 10 represents schematics of a transdermal drug delivery patch proposed in accordance with this invention.

### DETAILED DESCRIPTION OF THE INVENTION

Fundamental Theory on Principals Underlying the Invention

Effects of hydration on skin permeability have been extensively studied (5-11). Skin's mechanical properties (elasticity and plasticity) as well as permeability are known to be strongly dependent on the relative water content or hydration state of the SC (5,7-9,11). Although mechanisms of hydration-mediated permeability are not fully clear, swelling of stratum corneum and fluidization of lipid bilayers are believed to be responsible for this phenomenon (9,10). Absorption of water by the skin depends on its prior hydration state. Increase in the stratum corneum thickness by as much as 26% due to water absorption has been reported (6). Increase in skin volume should induce internal stresses, especially in keratin fibers, which need to enlarge to accommodate absorbed water. Under typical permeation experiments, where >1 cm² skin area is used, this stress is of little consequence since it is spread over a large area, thereby lowering the stress gradient. However, when a higher lateral gradient in the degree of hydration is induced, it may lead to substantial effects on skin structure and permeability. This study aims at studying the consequence of this hydration gradient.

A steep lateral gradient in hydration exists near the edge, that is, at the interface of the skin area that contacts the formulation and the area that does not. Specifically, the skin exposed to the formulation (say, PBS) swells due to the absorption of the formulation. Since the skin is expected to swell uniformly through the exposed area, swelling is unlikely to cause significant local stresses in the bulk skin. However, the skin that is not exposed to the formulation (for example, the skin that lies underneath the flange of the diffusion cell) is expected to be at a lower state of solvation (hydration). Thus, there exists a sharp hydration gradient at the interface between these two regions, which leads to a sharp gradient in skin expansion. We propose that this gradient induces local stresses that alter the skin structure at the cellular level, thereby enhancing skin permeability. The proposed mechanism is completely different than the “classical edge damage” hypothesis, which assumes that the damage caused by clamping of the skin damages the skin due to mechanical pressure (12,13,14). As will be shown later, we designed an experimental system that does not apply mechanical stresses on the skin, but rather creates a lateral hydration gradient.

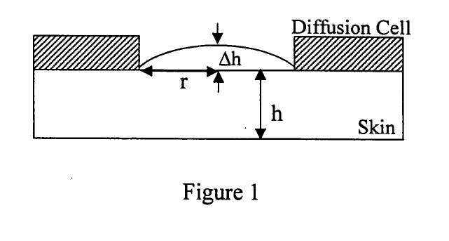

Consider a flat, circular piece of skin of full thickness h, placed in a diffusion cell of radius r, as shown in FIG. 1. Upon fully hydrating, the skin swells and its thickness increases by Δh. The skin, which was flat prior to hydration, now assumes the shape of an oblate spheroidal cap, defined by major axis r and minor axis Δh. The surface area of hydrated skin is given by the following equation.

$${A}_{hydrated} = π ( {r}^{2} + \frac{Δ {h}^{2}}{2 e} ln ( \frac{1 + e}{1 - e} ) ) \qquad [ 1 ]$$

where, e, is the eccentricity of the spheroid and is given by the following equation.

$${ⅇ}^{2} = 1 - \frac{Δ {h}^{2}}{{r}^{2}} \qquad [ 2 ]$$

Noting that the skin area prior to hydration was πr², the change in SC area upon hydration, ΔA, is given by the following equation.

$$Δ A = {A}_{hydrated} - {A}_{unhydrated} = π \frac{Δ {h}^{2}}{2 e} ln ( \frac{1 + e}{1 - e} ) \qquad [ 3 ]$$

The strain (percent increase in area) induced in the SC due to hydration, ε, is then given by the following equation.

$$ɛ = \frac{Δ A}{{A}_{unhydrated}} = \frac{1}{2 e} {( \frac{Δ h}{r} )}^{2} ln ( \frac{1 + e}{1 - e} ) \qquad [ 4 ]$$

Since the strain, ε, corresponds to the change in surface area of the skin, it essentially indicates the elongation experienced by the stratum corneum. Note that Δh is the change in skin thickness due to hydration and is an intrinsic characteristic of the skin. That is, Δh does not depend on skin area. This can be shown with the help of a simple mathematical treatment of the oblate spheroidal cap. Specifically, the volume of the swollen skin is given by the following equation.

$${V}_{cap} = \frac{2}{3} π {r}^{2} Δ h + π {r}^{2} h \qquad [ 5 ]$$

where, πr²h is the volume of skin prior to hydration. The increase in skin volume due to hydration can be calculated as follows:

$$Δ V = {V}_{hydrated} - {V}_{unhydrated} = \frac{2}{3} π {r}^{2} Δ h \qquad [ 6 ]$$

The change in skin volume per unit area, γ, is then given by the following equation.

$$γ = ( \frac{Δ V}{A} ) = ( \frac{Δ V}{π {r}^{2}} ) = \frac{2}{3} Δ h \qquad [ 7 ]$$

Thus Δh is a representative of change in skin volume per unit area on hydration, which is an intrinsic character of skin. It simply describes the capacity of skin to absorb water. Then, γ is just a physical constant that can be determined experimentally. For this purpose, we hydrated pig skin possessing different areas in the range of 0.36 cm² to 20 cm² with PBS. Skin was typically 4 mm thick. The skin was thawed at room temperature in open air for 24 hours and its mass, mₒ, was measured. The skin was then hydrated in excess phosphate buffered saline (PBS) for 24 hours and its mass, mₕ, was measured again. The amount of water absorbed by the skin, Δm, was calculated (Δm=mₕ−mₒ). The change in skin volume per unit area, γ, was then calculated using the following equation, γ=Δm/ρA, ρ being the density of PBS (1 g/cm³). Over the range of skin areas studied, γ was indeed found to be constant (data not shown) and was found to be 0.58±0.1 mm. This value of γ was used to calculate Δh which was found to be 0.87 mm.

Given that Δh is a constant, Eq. [4] predicts that the strain induced by hydration varies inversely with the area of the skin. For large skin areas (r˜1 cm, that is r>>Δh), the hydration strain, ε, is relatively small and may be non-consequential to transdermal drug transport. However, for smaller values of r, the strain induced by hydration can be significant and may affect transdermal drug transport. Increased strain is expected to increase skin permeability through structural alterations. Accordingly, skin permeability, P, is expected to increase with a decrease in the radius of its contact with the formulation, r.

$$P = F ( ɛ ) = F ( \frac{Δ {h}^{2}}{{r}^{2}} ) \sim f ( \frac{1}{r} ) \qquad [ 8 ]$$

where, f is a function whose exact dependence cannot be determined from the first principles at this point. Besides of fundamental interest, inverse dependence of skin permeability on contact area also has practical implications. Specifically, we predicted that an array of reservoirs should deliver more drug compared to that from a single reservoir. Although the contact area is reduced by using an array, skin permeability underneath each reservoir in the array is expected to be higher than that observed from a single large reservoir. If the fraction of the total skin area occupied by the reservoirs in the array is, α, then the effective permeability induced by the array, P_{array}, is given by the following equation.
P_{array}=αP [9]
where, P is permeability of skin underneath each reservoir. Depending on the values of α and P significant enhancements of skin permeability can be obtained.

Details

The amount of drug delivered by conventional patches can be assessed using a Franz diffusion cell. A Franz diffusion cell consists of a donor compartment and a receiver compartment, which is provided with a sampling arm. A skin piece is clamped between the donor and receiver compartment. The drug whose diffusion across the skin is to be studied is placed in the donor compartment. The drug solution uniformly contacts the skin and mimics the effect of a conventional transdermal patch. Over a period of time samples are obtained from the receiver compartment and are analyzed for the amount of drug that has crossed over from the donor into the receiver compartment.

The present invention is a novel transdermal drug delivery patch with an array of spatially organized reservoirs. In order to verify the validity of our invention we used three different assemblies, simulating the proposed matrix patch, that in essence provide the same working principle. All experiments were performed using pig skin. Skin was harvested from pigs (15) and was stored at −70° C. until the time of experiments. Skin was thawed at room temperature just before using it for experiments. A brief discussion of the experimental system used for the three cases follows.

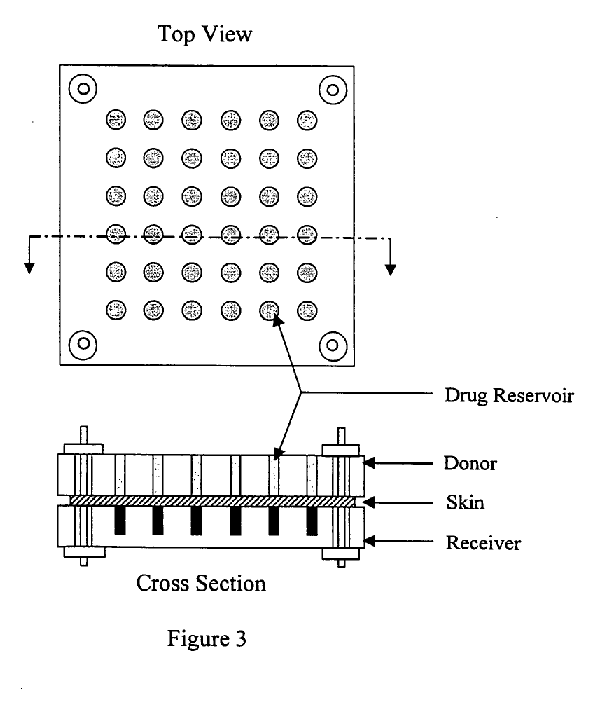

I. Polycarbonate/Teflon Array Assembly: This assembly consists of two plates made of polycarbonate/teflon—a donor plate and a receiver plate. The top plate (donor plate) has through holes (wells) drilled in it, each of which acts as an isolated donor chamber. The bottom plate (receiver plate) also has holes (wells) drilled in the same pattern as the donor plate and simulates the receiver compartment. The skin is placed between the donor and receiver plate and the plate assembly is clamped using four screws. This simple arrangement provides for a quick and efficient way of simulating an array with varying reservoir sizes. FIG. 3 is a schematic of the distributed array template. The receiver and donor plates are each 0.5 inches thick. Such array templates were used at varying reservoir sizes. Specifically, 4 different well diameters were used, 5 mm, 7 mm, 9 mm and 12 mm. Screening of the formulations was performed using pigskin. The wells in the receiver plate were filled with phosphate buffered saline (PBS). The skin was placed on the receiver plate with the stratum corneum facing the donor plate. The donor plate was then placed on the skin and the entire assembly was clamped tightly using four screws. A mild vacuum was then applied to remove any excess PBS that may be pushed in to the wells in the receiver plate.

Radiolabeled mannitol (³H) (American Radiolabeled Chemicals, St. Louis, Mo.) along with a model chemical enhancer sodium lauryl sulfate (SLS) (Fisher Scientific, Fairlawn, N.J.) was used as a model drug. A solution of 10 μL/mL (10 μCi/mL) of radiolabeled mannitol in a 0.5% solution of sodium lauryl sulfate in PBS was prepared. This solution was filled in all the wells in the donor plate of the array. Several wells of the same diameter were filled to get repetitions for statistical purposes. The skin was incubated in contact with these formulations for 24 hours. At the end of 24 hours samples were drawn from the receiver plate and analyzed in a liquid scintillation counter (Packard Tri-Carb 2100TR, Packard Instrument Company, Meriden, Conn.). Using these data the total radioactivity crossing the skin from the donor to the receiver compartment was calculated. Similar experiments were repeated with the Franz diffusion cell (PermeGear Inc., Bethlehem, Pa.), donor well diameter 16 mm, with the same model drug. The data was then analyzed to verify any effect of a distributed dispensing system on the amount of drug delivered across the skin.

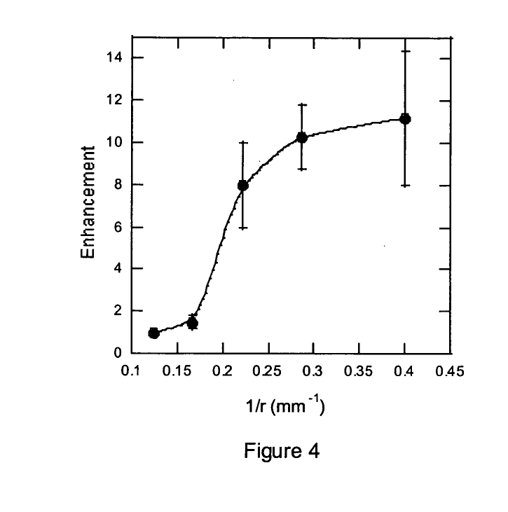

FIG. 4 shows the amount of mannitol delivered across the skin per unit area of the skin as a function of the size, r (mm), of the reservoir comprising the array. The data point corresponding to a reservoir size of 8 mm represents a single continuous reservoir that mimics a conventional transdermal patch. The amount delivered across the skin in this single reservoir lies at the lowest end of the curve and all other points are normalized with respect to this point. As the contact area is divided into a number of isolated reservoirs, the amount delivered into the skin through the array increases systematically reaching a value exceeding 10-fold as the reservoir size approached 2.5 mm.

Several investigators in the past have claimed that the observed effect is an artifact of physical damage to the skin at the edge of the reservoir due to the stress forces exerted by the clamping mechanism. In order to conclusively exhibit that the observed effect is in fact due to the differential hydration gradient created along the periphery of the reservoirs we repeated our experiments by using the following two assemblies (II and III). These assemblies have been designed to exclusively eliminate any mechanical stress on the skin.

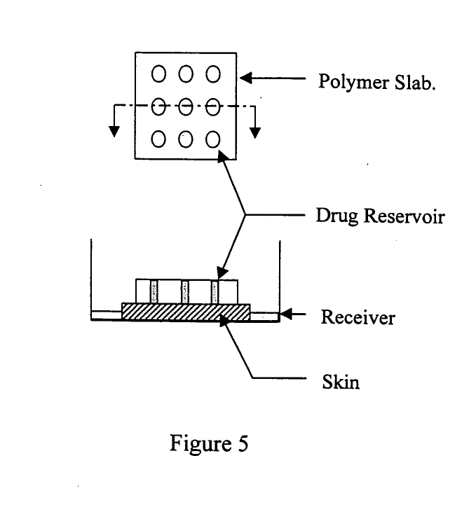

II. Polymer (Polyurethane) Array Assembly: Skin was placed in a PBS receiver fluid (10 ml) such that PBS contacted the skin on all sides and the bottom but not the top (FIG. 5). An array of liquid reservoirs was created by punching circular holes in a matrix pattern in a polyurethane slab (thickness=5 mm). These holes were separated from each other by one diameter. Such arrays were made for different size reservoirs (diameters in the range of 2-6 mm). The top surface of the skin was dried of any surface moisture. The “array template” was then mounted on the skin using an adhesive. Thus, no clamping was required to attach the reservoirs to the skin.

Transdermal transport experiments were performed using mannitol as a model solute and sodium lauryl sulfate (SLS) as a model enhancer. 10 μCi/ml of ³H labeled mannitol added to a solution of 0.5% SLS in PBS was added to all reservoirs. Excellent sealing was obtained between the polyurethane array and the skin and no leakage of formulations was observed. The reservoir array was placed on the skin for 24 hrs. The receiver compartment was sampled at the end of 24 hrs to calculate the amount of mannitol delivered across the skin into the PBS. Skin permeability in the skin exposed to reservoirs was calculated for each reservoir size by dividing the total amount delivered by the number of reservoirs, area of each reservoir, contact time, and mannitol concentration in each reservoir. Enhancement factors corresponding to various reservoir sizes were then calculated with reference to the permeability obtained from the largest reservoir (6 mm).

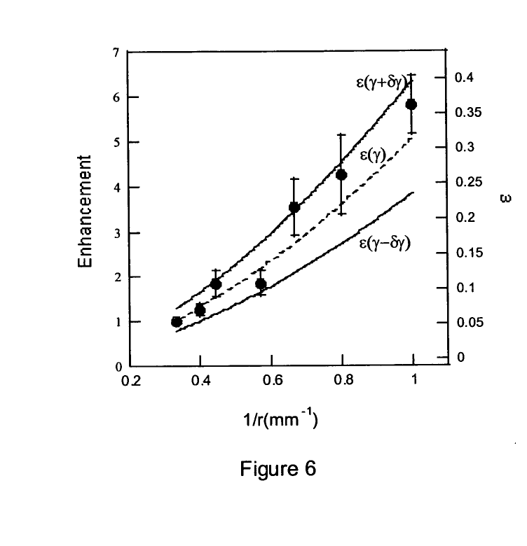

FIG. 6 shows the dependence of mannitol permeability enhancement measured in polyurethane arrays as a function of reciprocal of reservoir size, 1/r for seven different reservoir sizes (6 mm, 5 mm, 4.5 mm, 3.5 mm, 3 mm, 2.5 mm and 2 mm). The enhancements are calculated with respect to the permeability obtained from a 6 mm reservoir. The Figure also shows values of ε predicted by Eq. [4] (dotted line). It can be seen that the permeability enhancement increases with inverse reservoir size. The overall dependence of enhancement on 1/r is comparable to that of ε. Note that a comparison of the dependence of enhancement on 1/r with that of ε on 1/r should be performed at a qualitative level since no quantitative relationship between enhancement and ε is proposed at this point. It is however interesting to note that the uncertainty in the enhancement factors scales as the uncertainty in calculating theoretically the strain in the skin based on experimentally calculated γ. In other words this variability in calculating enhancement and strain arises from the error in measuring, experimentally, parameters that are related. We believe that the variability in enhancement comes from the local variability in the skin structure.

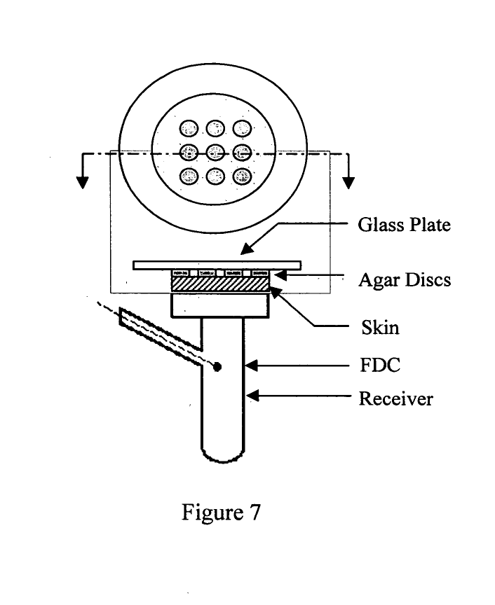

III. Agar Gel Disc Assembly: A third experimental system was used to assess the dependence of permeability on contact area. In this system, permeation of mannitol from gel disks was studied. For this purpose, agar gel disks were prepared by dissolving 0.5 gm agar (Becton Dickinson Microbiology Systems, Sparks, Md.) in 20 ml of 0.5% solution (w/v) SLS in PBS. 10 μCi/ml ³H radiolabeled mannitol was added to the mixture. The mixture was heated until agar formed a viscous solution. The viscous mixture was allowed to settle into a gel by pouring it into a petri dish to form a circular disc of 3.5 mm thickness. Disks of various diameters (16, 9, 5, and 3 mm) were then cut using a punch. These disks were then placed in an array pattern on the skin placed atop the Franz Diffusion Cell. A steel mesh was placed underneath the skin for support. A glass cover slide was placed above the disks to ensure good contact of skin with the disks. A schematic of this assembly is depicted in FIG. 7. Receiver compartments were sampled over 24 hrs and concentration of radiolabeled mannitol in these samples was measured using a scintillation counter. Permeability was calculated for each reservoir size by dividing the total flux obtained by the number of reservoirs at that particular size. Enhancement factors were calculated by dividing the permeability obtained from a disk of a particular size with reference to permeability obtained from the largest disk (16 mm).

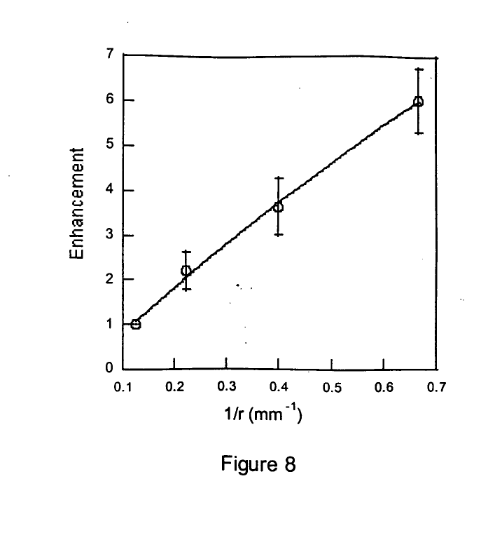

FIG. 8 shows the dependence of mannitol permeability enhancement measured in the gel disk array for varying reservoir sizes (16 mm, 9 mm, 5 mm and 3 mm). Once again, a significant increase in permeability is observed with a decrease in disk diameter. The overall dependence of enhancement on reservoir size is comparable to earlier systems, thus confirming that the dependence of permeability on contact area is not an artifact of any particular experimental system. The actual magnitudes of enhancements from these two systems should not be compared to each other since they have been normalized with respect to different size reservoirs. Furthermore, the lateral hydration gradient created in the skin in these two assemblies is likely to be different. Specifically, the hydration gradient is likely to be higher in the liquid reservoir arrays compared to that in the agar disk array.

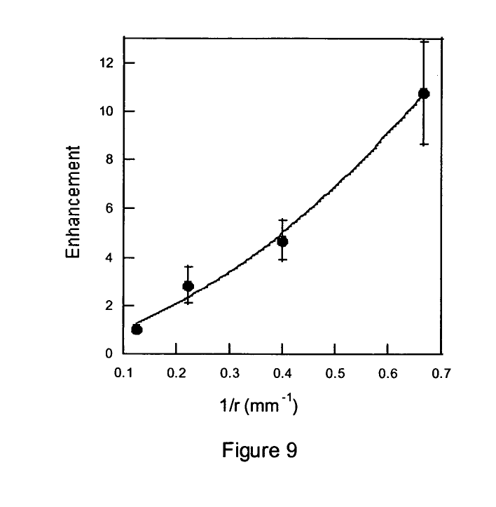

To experimentally determine the enhancement obtained by reservoir arrays, we created patches holding reservoir arrays containing ³H labeled mannitol as the model solute. These patches were created at varying reservoir sizes (16 mm, 9 mm, 5 mm and 3 mm). In these patches, the reservoirs were arranged in a square pattern with the center-to-center distance between two adjacent reservoirs set to twice the reservoir diameter. The patches were placed on skin pieces possessing an area of 10.25 cm². Keeping the area fraction the same for all the reservoir sizes (˜20%) drug reservoirs were placed on these skin pieces. With this configuration, the number of reservoirs that could be fitted in 10.25 cm² are 1, 4, 10, and 28 for reservoirs possessing diameters of 16 mm, 9 mm, 5 mm and 3 mm, respectively. The packing fraction used is not necessarily the maximum or optimum packing fraction, but is chosen simply to demonstrate the principles. FIG. 9 shows the amount of mannitol delivered per unit macroscopic area (i.e. 10.24 cm²) from arrays of various diameter reservoirs. A 3 mm reservoir array delivers about 11 times more mannitol that that from a 16 mm reservoir containing an identical formulation. Once again, the actual magnitude of the enhancement depends on the size and geometry of the skin used in the experiments. However, the data shown in FIG. 9 confirm that an array of reservoirs enhances skin permeability compared to that obtained from a single large reservoir.

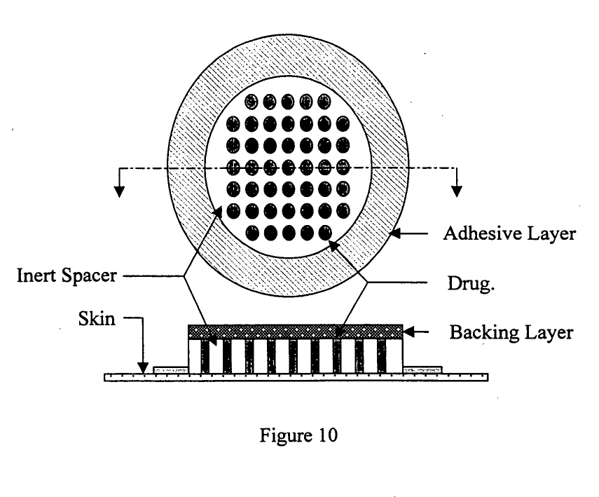

FIG. 10 shows the design of a patch based on the invention. The patch consists of an array of isolated drug-containing reservoirs. The space between the reservoirs can be either left empty or filled with an inert material to ensure isolation of the reservoirs. The drug matrix in the patch is provided with a backing layer of a impervious membrane to prevent outward diffusion of the drug from the reservoirs. An adhesive layer holds the patch on the skin. This patch is then placed on the skin such that the reservoirs contact the skin.

Although the present invention and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, composition of matter, means, methods and/or steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the invention is intended to include within its scope such processes, machines, manufacture, compositions of matter, means, methods, or steps.

### REFERENCES

(1) N. Kannikannan., K. Kandimalla., S. S. Lamba. and M. Singh. Structure-activity relationship of chemical penetration enhancers in transdermal drug delivery. Curr. Med. Chem. 7(6): 593-608 (2000).
(2) M. V. Chaubal. New opportunities in transdermal delivery systems. SPECTRUM: Drug delivery and reformulation technologies. Decision Resources Inc. Oct. 18, 2001.
(3) Bouwstra. J. A., The Skin, a Well Organized Membrane. Colloids and Surfaces A. 123-124: 403-413 (1997).
(4) Mitragotri, S. Synergistic effect of enhancers for transdermal drug delivery. Pharm. Res. 17 (11): 1354-1359, 2000.
(5) Papir. S. Y., Hsu. K. and Wildnauer. H. R. The mechanical properties of stratum corneum. I. The effect of water and ambient temperature on the tensile properties of newborn rat stratum corneum. Biochim. Biophys. Acta. 399: 170-180 (1975).
(6) Lars. N., Emilson. A. and Forslind. B. Stratum corneum swelling. Biophysical and computer assisted quantitative assessments. Arch. Dermatol. Res. 289: 506-513(1997).
(7) Barry. F. and Duzee. V. The influence of water content, chemical treatment and temperature on Theological properties of skin. J. Invest. Dermatol. 71(2): 140-144 (1978).
(8) B. E. Gregor., M. D. Jemec., B. Jemec., B. I. E. Jemec. and J. Serup. The effect of superficial hydration on mechanical properties of skin. Plast. Reconstr. Surg. 85(1):100-103 (1990).
(9) A. Alonso., N. C. Meirelles., V. E. Yushmanov., and M. Tabak. Water increases the fluidity of intercellular membranes of stratum corneum: Correlation with water permeability, elastic and electrical resistance properties. J. Invest. Dermatol. 106(5): 1058-63 (1996).
(10) T. Yamamura. and T. Tezuka. The water-holding capacity of the stratum corneum measured by ¹H-NMR. J. Invest. Dermatol. 93(1): 160-164 (1989).
(11) L. K. Pederson. and G. B. E. Jemec. Plasticising effect of water and glycerin on human skin in vivo. J. Dermatol. Sci. 19(1): 48-52 (1999).
(12) Walser. M. Role of edge damage in sodium permeability of toad bladder and a means of avoiding it. Am. J. Phs. 219 (1): 252-255 (1970).
(13) Helman. I. S. and Miller. D. A. Edge damage effect on measurement of urea and sodium flux in frog skin. Am. J. Phys. 226(5): 1198-1203 (1974).
(14) Helman. I. S. and Miller. D. A. Edge damage effect on Electrical measurements of frog skin. Am. J. Phys. 225(4): 972-977 (1973).
(15) S. Mitragotri., J. Farrell., T. Terahara., J. Kost. and R. J. Langer. Determination of threshold energy dose for ultrasound-induced transdermal drug delivery. J. Contr. Rel. 63:41-52 (2000).

1. A device for transdermal drug delivery, comprising a skin patch having a plurality of drug reservoirs arranged in a matrix, serving to divide the area of contact with the skin into a plurality of smaller areas.

2. A method of delivering a drug, comprising applying the drug transdermally to a plurality of locations arranged in a matrix on the skin of a recipient.

3. The method of claim 2 in which the drug is applied by means of a skin patch having a plurality of drug reservoirs arranged in a matrix, serving to divide the area of contact with the skin into a plurality of smaller areas.
